(19) United States
(12) Patent Application Publication
(10) Pub. No.: US 2011/0217202 A1
(43) Pub. Date: Sep. 8, 2011

(54) ACTIVE OXYGEN DISINFECTION SYSTEM AND USE THEREOF

(76) Inventor: Lynn Cook Winterton, Alpharetta, GA (US)

(21) Appl. No.: 13/041,466

(22) Filed: Mar. 7, 2011

Related U.S. Application Data
(60) Provisional application No. 61/311,587, filed on Mar. 8, 2010.

(51) Int. Cl. A61L 2/08; A01N 43/90; A01N 43/84; B65D 81/24

(52) U.S. Cl. 422/22; 514/333; 514/224.8; 514/455; 206/205

(57) ABSTRACT

The present invention provides a lens care kit for disinfecting and/or cleaning contact lenses. The lens care kit of the invention comprising: (1) a lens care solution; (2) a lens case for holding the lens care solution and a contact lens immersed in the lens care solution; (3) a singlet oxygen-generating agent which is dissolved or dispersed in a lens care solution; and (4) a light irradiation source for irradiating the singlet oxygen-generating agent for a period of time sufficient to produce a sufficient amount of singlet oxygen in the lens care solution to disinfect the contact lens.

This application claims the benefit under 35 U.S.C. §119 (e) of U.S. provisional application Ser. No. 61/311,587 filed on Mar. 8, 2010, herein incorporated by reference in its entirety.

This invention relates generally to a method, a solution, and a kit useful for cleaning and disinfecting a contact lens.

### BACKGROUND OF THE INVENTION

Contact lenses provide a means for vision correction for a wide range of consumers. The advantages of contact lens wear are numerous. Improved convenience and improved appearance in comparison to spectacle glasses are probably the two most important advantages to most consumers. However, contact lenses require stringent care regimes in order to ensure comfort and avoid ocular infections. Proper care of contact lenses typically requires the consumer to periodically clean and disinfect the lenses; thus preventing infection or other deleterious effects on ocular health which may be associated with contact lens wear.

One currently marketed lens care system is the use of multiple-purpose solutions to clean, disinfect, and rinse contact lenses without mechanically rubbing lenses. These new ‘multipurpose’ systems currently dominate the lens care market. Such popularity is likely derived from the ease and convenience provided by these new systems to consumers. In order to achieve satisfactorily disinfecting results, a contact lens has to be in a MPS solution for a sufficient time period. But, patients do not have a direct way to determine if their lenses have been in the lens care solution long enough to disinfect the lenses.

Another lens care system is the use of hydrogen peroxide solution as described in U.S. Pat. No. 4,585,488, U.S. Pat. No. 4,748,992, U.S. Pat. No. 4,812,173, U.S. Pat. No. 4,889,689, U.S. Pat. No. 4,899,914, U.S. Pat. No. 5,011,661, U.S. Pat. No. 5,275,784, U.S. Pat. No. 5,302,352, U.S. Pat. No. 5,468,448, U.S. Pat. No. 5,523,012, U.S. Pat. No. 5,196,174, U.S. Pat. No. 5,089,240, U.S. Pat. No. 5,558,846, U.S. Pat. No. 5,576,028, U.S. Pat. No. 5,609,264, U.S. Pat. No. 5,609,837, U.S. Pat. No. 5,756,044, U.S. Pat. No. 5,807,585, U.S. Pat. No. 5,958,351, U.S. Pat. No. 6,210,639, U.S. Pat. No. 6,440,411, U.S. Pat. No. 6,569,824, U.S. Pat. No. 6,945,389 and in copending U.S. patent application 61/261,844 filed 17 Nov. 2009 and 61/262,674 filed 19 Nov. 2009, herein incorporated by references in their entireties. However, one disadvantage of such a lens care system is that the hydrogen peroxide in the solution with contact lenses must be substantially decomposed or removed by other means, such as serial dilution or extraction, before the contact lenses can be comfortably inserted into the eyes of a patient.

A commonly-owned PCT patent application publication No. WO2008/021349 discloses a lens care system which comprises a colored lens care solution (a multipurpose solution or a hydrogen peroxide solution), a lens case having a singlet oxygen-generating agent covalently attached to the solution-contacting surface of the lens case, and a light source for gradually decomposing colorants in the colored lens care solution and rendering the colored lens care solution colorless over a specific time period, thereby indicating that lenses under disinfecting and cleaning by the colored lens care solution are ready for use. Methods for disinfecting contact lenses disclosed in WO2008/021349 are still based on either multipurpose solutions or hydrogen peroxide solutions.

Thus, it would be desirable to provide a new lens care system for disinfection of contact lenses.

### SUMMARY OF THE INVENTION

Generally described, the present invention provides a lens care system (or kit) for the cleaning and disinfecting of contact lenses, comprising: (1) a lens care solution; (2) a lens case for holding the lens care solution and a contact lens immersed in the lens care solution; (3) a singlet oxygen-generating agent which is dissolved or dispersed in a lens care solution and/or covalently attached onto a surface of the lens case in contact with the lens care solution; and (4) a light irradiation source for irradiating the singlet oxygen-generating agent for a period of time sufficient to produce a sufficient amount of singlet oxygen in the lens care solution to disinfect the contact lens.

The present invention also provides a lens care solution for disinfecting and/or cleaning contact lenses, comprising one or more oxygen-generating agents dissolved or dispersed in an aqueous solution.

The present invention further provides a method for disinfecting and/or cleaning contact lenses using a lens care system of the invention.

The present invention provides the foregoing and other features, and the advantages of the invention will become further apparent from the following detailed description of the example embodiments set forth herein. The detailed description is merely illustrative of the invention and does not limit the scope of the invention, which is defined by the appended claims and equivalents thereof.

### DETAILED DESCRIPTION OF PREFERRED EMBODIMENTS

The present invention may be understood more readily by reference to the following detailed description of the invention which forms a part of this disclosure. Unless defined otherwise, all technical and scientific terms used herein have the same meaning as commonly understood by one of ordinary skill in the art to which this invention belongs. Generally, the nomenclature used herein is well known and commonly employed in the art. Conventional methods are used for carrying out the disclosed procedures, such as those provided in the art and various general references. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, reference to singular forms such as “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The invention, in one aspect, provides a lens care kit (system) for cleaning and disinfecting contact lenses. A lens care kit (system) of the invention comprises: (1) a lens care solution; (2) a lens case for holding the lens care solution and a contact lens immersed in the lens care solution; (3) a singlet oxygen-generating agent which is dissolved or dispersed in a lens care solution and/or covalently attached onto a surface of the lens case in contact with the lens care solution; and (4) a light irradiation source for irradiating the singlet oxygen-generating agent for a period of time sufficient to produce a sufficient amount of singlet oxygen in the lens care solution to disinfect the contact lens.

A lens care kit of the invention can be used to disinfect and clean contact lenses including hard (PMMA) contact lenses, soft (hydrophilic) contact lenses, and rigid gas permeable (RGP) contact lenses. The soft contact lenses are hydrogel contact lenses or silicone hydrogel contact lenses.

For the purposes of the present invention the term “disinfect” means the rendering non-viable of substantially all pathogenic microbes that are in the vegetative state, including gram negative and gram positive bacteria, as well as fungi.

A “hydrogel” refers to a polymeric material which can absorb at least 10 percent by weight of water when it is fully hydrated. Generally, a hydrogel material is obtained by polymerization or copolymerization of at least one hydrophilic monomer in the presence of or in the absence of additional monomers and/or macromers.

A “silicone hydrogel” refers to a hydrogel obtained by copolymerization of a polymerizable composition comprising at least one silicone-containing vinylic monomer or at least one silicone-containing macromer.

“Hydrophilic,” as used herein, describes a material or portion thereof that will more readily associate with water than with lipids.

The lens care kit (system) of the invention allows customers to disinfect and clean contact lenses. The invention is relied on singlet oxygen to disinfect and clean contact lenses. Singlet oxygen is a highly reactive species which has been used in photodynamic therapy to kill cancer cells. It is discovered here that singlet oxygen can be used effectively to disinfect contact lenses. Singlet oxygen can also decompose deposits (such as proteins, lipids, etc.) on and in worn contact lenses and thereby facilitate removal of the deposits from the worn contact lenses.

The time period is sufficiently long for disinfecting of contact lenses. It can range from about 5 minutes to about 8 hours or longer, preferably up to about 6 hours, more preferably up to about 4 hours, even more preferably up to about one hour.

In accordance with the invention, a singlet oxygen-generating agent is intended to describe a compound or moiety capable of generating singlet oxygen under UV/visible light irradiation. Singlet oxygen-generating agents include photosensitizers as known to a person skilled in the art. Exemplary preferred singlet oxygen-generating agents include without limitation Rose Bengal, methylene blue, Azure A, various porphyrins and metalloporphyrins (e.g., zinc tetrahydroxyphenyl-porphyrin, zinc tetracarboxyphenylporphyrin, zinc uroporphyrin, zinc protoporhyrin, tetrasulphonatophenylporphyrin, Zn-tetrasulphonatophenylporphyrins, tetramethylpyridinium porphyrin, Zn-tetramethylpyridinium porphyrins, haematoporphyrin, Zn-haematoporphyrin, or the like), various phthalocyanins and metallophthalocyanins (e.g., cationic water-soluble pyridinium Zn phthalocyanin, sulphonated phthalocyanins, sulphonated metallophthalocyanins, the likes), and combinations thereof.

In one embodiment, a singlet oxygen-generating agent is dissolved or dispersed in a lens care solution of the invention for disinfect contact lenses.

In a preferred embodiment, a singlet oxygen-generating agent is first modified by attaching it to a hydrophilic polymer having a molecular weight sufficient high so as to prevent the singlet oxygen-generating agent from being absorbed by lens material and then the hydrophilic polymer with the singlet oxygen-generating agent attached thereon is dissolved or dispersed in a lens care solution of the invention for disinfect contact lenses. The molecular weight of a hydrophilic polymer is from about 600 to 5,000,000 Daltons, preferably from about 2000 to 2,000,000 Daltons, more preferably about 5000 to 1,000,000 Daltons, even more preferably from about 10,000 to 1,000,000 Daltons.

For example, a polyethylene glycol urea (PEG-urea with a molecular weight of about 1000 to about 1,000,000 Daltons) or an amino-dextran (M.W.=about 100,000 to 1,000,000 Daltons) can be covalently attached to a singlet oxygen-generating agent (one described above or known to a person skilled in the art) through a known coupling agent based on a known coupling reaction. A person skilled in the art will know how to covalently attach a polymer onto a singlet oxygen-generating.

It is well known in the art that coupling reactions between a pair of matching functional groups can be used to form covalent bonds or linkages under various reaction conditions well known to a person skilled in the art, such as, for example, oxidation-reduction conditions, dehydration condensation conditions, addition conditions, substitution (or displacement) conditions, Diels-Alder reaction conditions, cationic crosslinking conditions, ring-opening conditions, and epoxy hardening conditions.

Non-limiting examples of coupling reactions between a pair of matching co-reactive functional groups selected from the group preferably consisting of amino group (—NHR in which R is hydrogen or a C₁-C₂₀ unsubstituted or substituted, linear or branched alkyl group), hydroxyl group, carboxylic acid group, acid halide groups (—COX, X═Cl, Br, or I), acid anhydrate group, aldehyde group, azlactone group

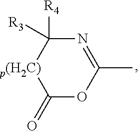

in which p is 0 or 1; R₃ and R₄ independently can be an alkyl group having 1 to 14 carbon atoms, a cycloalkyl group having 3 to 14 carbon atoms, an aryl group having 5 to 12 ring atoms, an arenyl group having 6 to 26 carbon and 0 to 3 sulfur, nitrogen and/or oxygen atoms, or R₃ and R₄ taken together with the carbon to which they are joined can form a carbocyclic ring containing 4 to 12 ring atoms), isocyanate group, epoxy group, aziridine group, and amide groups (—CONH₂), are given below for illustrative purposes. An amino group reacts with aldehyde group to form a Schiff base which may further be reduced; an amino group —NHR reacts with an acid chloride or bromide group or with an acid anhydride group to form an amide linkage (—CO—NR—); an amino group —NHR reacts with an isocyanate group to form a urea linkage (—NR—C(O)—NH—); an amino group —NHR reacts with an epoxy or aziridine group to form an amine bond (C—NR); an amino group reacts (ring-opening) with an azlactone group to form a linkage (—C(O)NH—CR₃R₄—(CH₂)p-C(O)—NR—); an amino group —NHR reacts with a carboxylic acid group in the presence of a coupling agent—carbodiimide (e.g., 1-ethyl-3-(3-dimethylaminopropyl) carbodiimide (EDC), N,N′-dicyclohexylcarbodiimide (DCC), 1-cyclohexyl-3-(2-morpholinoethyl)carbodiimide, diisopropyl carbodiimide, or mixtures thereof) to form an amide linkage; a hydroxyl reacts with an isocyanate to form a urethane linkage; a hydroxyl reacts with an epoxy or aziridine to form an ether linkage (—O—); a hydroxyl reacts with an acid chloride or bromide group or with an acid anhydride group to form an ester linkage; an hydroxyl group reacts with an azlactone group in the presence of a catalyst to form a linkage (—C(O)NH—CR₃R₄—(CH₂)p-C(O)—O—).

It is also understood that coupling agents with two functional groups may be used in the coupling reactions. For example, a diisocyanate or di-acid halide, di-carboxylic acid, di-azlactone, di-epoxy or di-aziridine compound can be used in the coupling of two hydroxyl or amino groups or combination thereof; a diamine or dihydroxyl compound can be used in the coupling of two isocyanate, epoxy, aziridine, carboxylic acid, acid halide or azlactone groups or combinations thereof.

In another preferred embodiment, particles having one or more singlet oxygen-agent are dispersed in a lens care solution of the invention for disinfecting contact lenses. Particles can be made of one or more inorganic materials or of one or more polymeric materials. A person skilled in the art will know well how to prepare particles having functional groups thereon and how to covalently attach one or more singlet oxygen-generating agents onto particle surfaces through surface functional groups based on a coupling reaction as discussed above. The particles has an average size of less than about 1 millimeter in diameter, preferably less than about 500 micrometers in diameter, more preferably less than about 100 micrometers in diameter, even more preferably less than about 1 micrometer in diameter.

Singlet oxygen-generating agents can be incorporated/distributed in particles, or preferably covalently attached onto the surfaces of the particles.

It is desired that singlet oxygen-generating agents are not adsorbed by a contact lens. If adsorbed, it needs to be rendered inactive, washed away, or be of such low concentration such that the adsorbed singlet oxygen-generating agent will not inadvertently generate irritating or toxic levels of singlet oxygen in the eye upon exposure to strong (light) irradiation. By covalently attaching singlet oxygen-generating agents either onto a hydrophilic polymer having a high molecular weight (from about 1000 to 5,000,000 Daltons, preferably from about 2000 to 2,000,000 Daltons, more preferably about 5000 to 1,000,000 Daltons, even more preferably from about 10,000 to 1,000,000 Daltons) or to the surface of particles, the adsorption of singlet oxygen-generating agents in the lens care solution of the invention by contact lenses can be minimized or eliminated.

In accordance with the invention, a lens care solution of the invention preferably is ophthalmic safe. The term “ophthalmically safe” with respect to a lens care solution is meant that a contact lens treated with the solution is safe for direct placement on the eye without rinsing, that is, the solution is safe and sufficiently comfortable for daily contact with the eye via a contact lens or direct installation. An ophthalmically safe solution has a tonicity and pH that is compatible with the eye and comprises materials, and amounts thereof, that are non-cytotoxic according to international ISO standards and U.S. FDA regulations.

The term “compatible with the eye” means a solution that may be in intimate contact with the eye for an extended period of time without significantly damaging the eye and without significant user discomfort.

A lens care solution of the invention is preferably formulated in such a way that it is essentially isotonic (osmolarity) with the lacrimal fluid, within a physiologically acceptable range of pH, and/or a desired viscosity.

A solution which is isotonic with the lacrimal fluid is generally understood to be a solution whose concentration corresponds to the concentration of a 0.9% sodium chloride solution (308 mOsm/kg).

A lens care solution of the invention preferably has at least one property selected from the group consisting of: a pH within a physiologically acceptable range of from about 6.0 to about 8.0, preferably about 6.5 to about 7.5, more preferably about 6.8 to about 7.3; a tonicity of from about 200 to about 450 milliosmol (mOsm), preferably from about 250 to 350 mOsm; a viscosity of from about 1.0 centipoise to about 20 centipoise at 25° C., preferably from about 1.5 centipoise to about 15 centipoise at 25° C., more preferably from about 2.0 centipoise to about 8 centipoise at 25° C.; and combinations thereof.

Deviations from the concentration ranges above are possible throughout, provided that the contact lenses to be treated are not damaged.

A lens care solution of the invention can have any combinations of the preferred embodiments of the pH, tonicity, and viscosity described above.

In accordance with the invention, a lens care solution of the invention can further comprises one or more components selected from the group consisting of one or more buffering agents, one or more lubricants, one or more conditioning/wetting agents, one or more viscosity-enhancing agents, one or more tonicity agents, one or more surfactants, one or more chelating agents, one or more microbicides/preservatives, and combinations thereof.

The solution of the present invention preferably contains a buffering agent. The buffering agents maintain the pH preferably in the desired range, for example, in a physiologically acceptable range of about 6.0 to about 8.0. Any known, physiologically compatible buffering agents can be used. Suitable buffering agents as a constituent of the contact lens care composition according to the invention are known to the person skilled in the art. Examples are boric acid, borates, e.g. sodium borate, citric acid, citrates, e.g. potassium citrate, bicarbonates, e.g. sodium bicarbonate, TRIS (trometamol, 2-amino-2-hydroxymethyl-1,3-propanediol), bis-aminopolyols, phosphate buffers, e.g. Na₂HPO₄, NaH₂PO₄, and KH₂PO₄ or mixtures thereof. The amount of each buffer agent is that amount necessary to be effective in achieving a pH of the composition of from about 6.5 to about 7.5. Typically, it is present in an amount of from 0.001% to 2%, preferably from 0.01% to 1%; most preferably from about 0.05% to about 0.30% by weight.

The preferred buffering agents are bis-aminopolyols of formula (I)

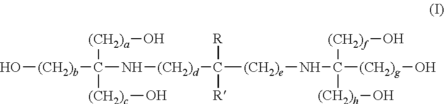

wherein a, b, c, d, e, f, g, and h are independently an integer from 1 to 6; and R and R′ are independently selected from the group consisting of —H, —CH₃, —(CH₂)₂₋₆—H, and —(CH₂)₁₋₆—OH. In the present invention, the buffering agents described by formula (I) may be provided in the form of various water-soluble salts. A most preferred bis-aminopolyol is 1,3-bis(tris[hydroxymethyl]methylamino)propane (bis-TRIS-propane) shown in formula II.

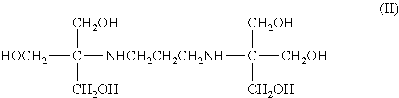

The dissociation constants for this dibasic compound are pKa₁=6.8 and pKa₂=9.5 which renders aqueous solutions of this compound useful as a buffering agent in a broad pH range from about 6.3 to 9.3. bis-TRIS-propane at a concentrations used in this invention is harmless to the eye and to known contact lens materials and is, therefore, ophthalmically compatible.

A lens care solution of the invention preferably also comprises a lubricant. “Lubricants” as used herein refer to any compounds or materials which can enhance surface wettability of a contact lens and/or the eye or reduce the frictional character of the contact lens surface. Examples of lubricants include without limitation mucin-like materials and hydrophilic polymers.

Exemplary mucin-like materials include without limitation polyglycolic acid, polylactides, collagen, and gelatin. A mucin-like material may be used to alleviate symptoms associated with dry eye syndrome. The mucin-like material preferably is present in effective amounts.

Any suitable hydrophilic polymers can be used so long as they are ophthalmically compatible. Exemplary hydrophilic polymers include, but are not limited to, polyvinyl alcohols (PVAs), polyamides, polyimides, polylactone, a homopolymer of a vinyl lactam, a copolymer of at least one vinyl lactam in the presence or in the absence of one or more hydrophilic vinylic comonomers, alkylated polyvinylpyrrolidones, a homopolymer of acrylamide or methacrylamide, a copolymer of acrylamide or methacrylamide with one or more hydrophilic vinylic monomers, poly(ethylene oxide) (PEO), a polyoxyethylene derivative, poly-N—N-dimethylacrylamide, polyacrylic acid, poly 2 ethyl oxazoline, heparin polysaccharides, polysaccharides, and mixtures thereof.

Examples of N-vinyl lactams include N-vinyl-2-pyrrolidone, N-vinyl-2-piperidone, N-vinyl-2-caprolactam, N-vinyl-3-methyl-2-pyrrolidone, N-vinyl-3-methyl-2-piperidone, N-vinyl-3-methyl-2-caprolactam, N-vinyl-4-methyl-2-pyrrolidone, N-vinyl-4-methyl-2-caprolactam, N-vinyl-5-methyl-2-pyrrolidone, N-vinyl-5-methyl-2-piperidone, N-vinyl-5,5-dimethyl-2-pyrrolidone, N-vinyl-3,3,5-trimethyl-2-pyrrolidone, N-vinyl-5-methyl-5-ethyl-2-pyrrolidone, N-vinyl-3,4,5-trimethyl-3-ethyl-2-pyrrolidone, N-vinyl-6-methyl-2-piperidone, N-vinyl-6-ethyl-2-piperidone, N-vinyl-3,5-dimethyl-2-piperidone, N-vinyl-4,4-dimethyl-2-piperidone, N-vinyl-7-methyl-2-caprolactam, N-vinyl-7-ethyl-2-caprolactam, N-vinyl-3,5-dimethyl-2-caprolactam, N-vinyl-4,6-dimethyl-2-caprolactam, and N-vinyl-3,5,7-trimethyl-2-caprolactam.

A very useful hydrophilic polymer is polyvinylpyrrolidone (PVP). The polyvinylpyrrolidone (PVP) used in the compositions of the invention is a linear homopolymer or essentially a linear homopolymer comprising at least 90% repeat units derived from 1-vinyl-2-pyrrolidone monomers, the polymer more preferably comprising at least about 95% or essentially all of such repeat units, the remainder selected from polymerization-compatible monomers, preferably neutral monomers, such as alkenes or acrylates. Other synonyms for PVP include povidone, polyvidone, 1-vinyl-2-pyrrolidinone, and 1-ethenyl-2-pyrolionone (CAS registry number 9003-39-8). Such materials are sold by various companies, including ISP Technologies, Inc. under the trademark PLASDONE™ K-29/32, from BASF under the trademark KOLLIDON™ for USP grade PVP, for example KOLLIDON™ K-15, K-30, K-60, K-90, K-120. While the invention is not limited to any specific PVP, K-90 PVP is preferred, more preferably pharmaceutical grade.

Examples of copolymers of n-vinylpyrrolidone with one or more vinylic monomers includes without limitation vinylpyrrolidone/vinylacetate copolymers, vinylpyrrolidone/dimethylaminoethylmethacrylate copolymers (e.g., Copolymer 845, Copolymer 937, Copolymer 958 from ISP Corporation), vinylpyrrolidone/vinylcaprolactam/dimethyl-aminoethylmethacrylate copolymer, and combinations thereof.

Examples of alkylated polyvinyl pyrrolidone copolymers include without limitation the family of GANEX® Alkylated polyvinyl pyrrolidone copolymer from ISP Corporation.

The number-average molecular weight Mₙ of the hydrophilic polymer is preferably from 5,000 to 5,000,000, more preferably from 10,000 to 1,000,000.

The solution may also contain one or more viscosity-enhancing agents. Suitable viscosity-enhancing components include, but are not limited to, polyvinylpyrrolidone, copolymer of N-vinylpyrrolidone and one or more hydrophilic vinylic monomers, water soluble natural gums, cellulose-derived polymers, and combinations thereof. Useful natural gums include guar gum, gum tragacanth and the like. Examples of useful cellulose-derived polymers as viscosity-enhancing agents include without limitation cellulose ethers.

Exemplary preferred cellulose ethers are methyl cellulose (MC), ethyl cellulose, hydroxymethylcellulose, hydroxyethyl cellulose (HEC), hydroxypropylcellulose, hydroxypropylmethyl cellulose (HPMC), or a mixture thereof. More preferably, a cellulose ether is hydroxyethyl cellulose (HEC), hydroxypropylmethyl cellulose (HPMC), and mixtures thereof. The cellulose ether is present in the composition in an amount of from about 0.01% to about 5% by weight, preferably from about 0.05% to about 3% by weight, even more preferably from about 0.1% to about 1% by weight, based on the total amount of contact lens care composition. It is believed that a cellulose ether can be used to increase the viscosity of a lens care and also can serve as a lubricant in the lens care composition.

In a preferred embodiment, the lens care solution in a lens care system of the invention comprises one or more components selected from the group consisting of polyvinylalcohol, polyvinylpyrrolidone, a vinylpyrrolidone/vinylacetate copolymer, a vinylpyrrolidone/dimethylaminoethylmethacrylate copolymer, a vinylpyrrolidone/acrylic acid copolymer, a vinylpyrrolidone/methacrylic acid copolymer, a vinylpyrrolidone/vinylcaprolactam/dimethyl-aminoethylmethacrylate copolymer, a vinylpyrrolidone/vinyl acetate copolymer with a given degree of hydrolysis (e.g., at least a degree of hydrolysis of at least about 70%, preferably at least about 80%, even more preferably at least about 90%), methyl cellulose (MC), ethyl cellulose, hydroxymethylcellulose, hydroxyethyl cellulose (HEC), hydroxypropylcellulose, hydroxypropylmethyl cellulose (HPMC), hyaluronic acid or salts thereof, carboxymethylcellulose, polyglycolic acid, polylactides, collagen, gelatin, xanthan gum, gum Arabic, starch, polyacrylic acid, polymethacrylic acid, copolymer of acrylamide and acrylic acid, and combinations thereof. All of the preferred embodiments of those components described above will be incorporated in this preferred embodiment. According to this preferred embodiment, the amount of any component(s) in a lens care solution of the invention is from about 0.01% to about 5% by weight, preferably from about 0.05% to about 3% by weight, even more preferably from about 0.1% to about 1% by weight, based on the total amount of contact lens care solution.

The isotonicity with the lacrimal fluid, or even another desired tonicity, may be adjusted by adding organic or inorganic substances (tonicity agents) which affect the tonicity. Suitable ophthalmically acceptable tonicity agents include, but are not limited to sodium chloride, potassium chloride, glycerol, propylene glycol, polyols, dexpanthenol, mannitols, xylitol, sorbitol, and mixtures thereof. Preferably, the tonicity of the solution is provided by one or more compounds selected from the group consisting of non-halide containing electrolytes (e.g., sodium bicarbonate) and non-electrolytic compounds. The tonicity of the solution is typically adjusted to be in the range from about 200 to about 450 milliosmol (mOsm), preferably from about 250 to 350 mOsm.

A lens care solution of the invention can also comprise one or more conditioning/wetting agents (e.g., polyvinyl alcohol, polyoxamers, polyvinyl pyrrolidone, hydroxypropyl cellulose, and mixture thereof).

In accordance with the invention the lens care solution can further comprise a surfactant for cleaning the contact lens. Any suitable known surfactants can be used in the invention. Examples of suitable surfactants include, but are not limited to homopolymers of polyethylene glycol or polyethyleneoxide, poloxamers under the tradename Pluronic from BASF Corp. (Pluronic™ and Pluronic-R™) which are nonionic surfactants consisting of block copolymers of propylene oxide and ethylene oxide; poloxamine which is a block copolymer derivative of ethylene oxide and propylene oxide combined with ethylene diamine; tyloxapol, which is 4-(1,1,3,3-tetramethylbutyl)phenol polymer with formaldehyde and oxirane; ethoxylated alkyl phenols, such as various surface active agents available under the tradenames TRITON (Union Carbide, Tarrytown, N.Y., USA) and IGEPAL (Rhone-Poulenc, Cranbury, N.J., USA); polysorbates such as polysorbate 20, including the polysorbate surface active agents available under the tradename TWEEN (ICI Americas, Inc., Wilmington, Del., USA.); alkyl glucosides and polyglucosides such as products available under the tradename PLANTAREN (Henkel Corp., Hoboken, N.J., USA); and polyethoxylated castor oils commercially available from BASF under the trademark CREMAPHOR; and combinations thereof.

Preferred surfactants include homopolymers of polyethylene glycol or polyethyleneoxide, and certain poloxamers such as materials commercially available from BASF under the tradenames PLURONIC® 17R4, PLURONIC® F-68NF, PLURONIC® F68LF, and PLURONIC® F127, with PLURONIC® F-68NF (National Formulary grade) being the most preferred. More preferably, a combination of PLURONIC® 17R4 and PLURONIC® F127 is used. When present, poloxamers may be employed at from about 0.001% to about 5% by weight, preferably from about 0.005% to about 1% by weight, more preferably from about 0.05% to about 0.6% by weight.

A lens care solution of the invention may include an effective amount of a chelating agent. Any suitable, preferably ophthalmically acceptable, chelating agents may be included in the present compositions, although ethylenediaminetetraacetic acid (EDTA), salts thereof and mixtures thereof are particularly effective. EDTA is low level non-irritating chelating agent and can be synergistic with PHMB to increase antimicrobial efficacy. Typical amount of EDTA is from about 0.001% to about 1% by weight, preferably from about 0.002% to about 0.5% by weight, more preferably from about 0.004% to about 0.1, even more preferably from about 0.005 to about 0.05, based on the total amount of contact lens care composition.

A lens care solution of the invention may include a preservative. Examples of preservatives include without limitation benzalkonium chloride and other quaternary ammonium preservative agents, phenylmercuric salts, sorbic acid, chlorobutanol, disodium edetate, thimerosal, methyl and propyl paraben, benzyl alcohol, and phenyl ethanol.

A lens care solution of the invention may contain a microbicide in a concentration sufficient to effect the desired disinfection of a contact lens. The specific concentrations required for the microbicides useful in this invention must be determined empirically for each microbicide. Some of the factors affecting the effective concentration are specific activity of the microbicide against the specified pathogens, the molecular weight of the microbicide, and the solubility of the microbicide. It is also important that the chosen microbicides be employed in a physiologically tolerable concentration. The list of microbicides which may be employed in the present invention include, but is not in limited to biguanides and salts thereof, biguanide polymers and salts thereof, a polyquaternium (which is a class of polycationic polymers, e.g., Polyquaternium-1 to Polyquaternium-47 according to the International Nomenclature for Cosmetic Ingredients designation), bronopol, benzalkonium chloride, hydrogen peroxide, and combinations thereof. The presently useful antimicrobial biguanides include biguanides, biguanide polymers, salts thereof, and mixtures thereof. Preferably, the biguanide is selected from alexidine free-base, salts of alexidine, chlorhexidine free-base, salts of chlorhexidine, hexetidine, hexamethylene biguanides, and their polymers, and salts thereof. Most preferably, the biguanide is a hexamethylene biguanide polymer (PHMB), also referred to as polyaminopropyl biguanide (PAPB).

Typically, the microbicides PHMB is present in a lens care solution in an amount of from about 0.01 to about 10 ppm, preferably from about 0.05 to about 5 ppm, more preferably from about 0.1 to about 2 ppm, even more preferably from about 0.2 to about 1.0 ppm.

Although PHMB has a broad spectrum of activity and non-specific mode of action against bacteria, PHMB might be able to cause some level of corneal staining (Lyndon Jones, et. al. “Asymptomatic corneal staining associated with the use of balafilcon silicon-hydrogel contact lenses disinfected with a polyaminopropyl biguanide-preserved care regimen”, Optometry and Vision Science 79: 753-61 (2002)). Therefore, it would be desirable to lower the amount of PHMB in a lens care solution while maintaining the antimicrobial efficacy of the lens care solution.

The lens care solutions according to the invention are produced in known manner, in particular by means of conventional mixing of the constituents with water or dissolving the constituents in water. Solvents for preparing a lens care solution of the invention can be water, a mixture of water or aqueous salt solution with a physiologically tolerable polar organic solvent, such as, for example, glycerol.

Any suitable lens cases can be used in the invention. One kind of lenses are containers used for disinfecting contact lenses based on hydrogen peroxide lens care systems can be used in the invention, such as, for examples, AOSEPT® Cup and those described in U.S. Pat. No. 5,089,240, U.S. Pat. No. 5,196,174, U.S. Pat. No. 5,275,784, U.S. Pat. No. 5,292,488, U.S. Pat. No. 5,520,227, U.S. Pat. No. 5,558,846, U.S. Pat. No. 5,609,264, U.S. Pat. No. 5,609,837, U.S. Pat. No. 5,756,044, U.S. Pat. No. 5,958,351, U.S. Pat. No. 6,210,639, and U.S. Pat. No. 6,569,824 (herein incorporated by references in their entireties). Another kind of lens cases are those known to a person skilled in the art that typically comprise a main body portion which includes a pair of separate and discrete wells (cavities or reservoirs) each adapted to receive one contact lens and an amount of a lens care solution. Each well has an open end having a substantially circular, oval or rain-drop shape periphery defining an opening. The lens case further comprises one or two caps adapted to be affixed to the wells at their open ends so as to provide a substantially liquid-impermeable seal. The caps each further include a sealing rim or surface adapted to mate with peripheries surrounding wells. The lens case may be constructed of a material which is sturdy and impervious to chemicals contained in a lens solution. For example, polystyrene, high-density polyethylene, or polypropylene can be the construction material of choice, although others may be used.

Preferably, a singlet oxygen-generating agent can be covalently attached to the solution-contacting surface of a lens case for treating contact lenses, or to the surface of a solid support, such as glasses, resins, or cloth tissues. A layer of a singlet oxygen-generating agent can be attached covalently onto a solid support or lens case by optionally first functionalizing the surface of the solid support or lens case (if there is no functional groups on the surface) to obtain function groups and then covalently attaching the layer of singlet oxygen-generating agent. Surface modification (or functionalization) of a solid support is well known to a person skilled in the art. Any known suitable method can be used.

Preferably, singlet oxygen agents can be bound covalently to the functionalized surface of a solid support or directly onto the functional groups on the surface of the solid support or directly onto the surface of a lens case according to any coupling reactions described above or any ones known to a person skilled in the art. Solid supports with singlet oxygen-generating agent covalently attached thereto can be placed in a well of a lens case for holding a contact lens and a given amount of a lens care solution.

In accordance with the invention, a light radiation source can be any light sources known to a person skilled in the art, so long as the light source can emit a light which can excite a singlet oxygen-generating agent to generate singlet oxygen. Preferred light source is light emitting device (LED). A LED would turn on inside the lens case after the lens case caps for the lens case are placed into place in a sealed state. A person skilled in the art will know well how to select a LED for a given singlet oxygen-generating agent.

The kit can optionally include instructions for how to use the lens care solution to clean and lubricate contact lenses directly in eyes.

The contact lens can be disinfected with a lens care system of the invention by immersing the lens in a lens care solution of the invention in a lens case. Although not necessary, the solution containing the contact lens can be agitated, for example, by shaking the lens case containing the solution and contact lens, to at least facilitate removal of deposit material from the lens.

A lens care kit (system) of the invention can be used to disinfect contact lenses against a wide range of microorganisms including but not limited to Fusarium solani, Staphylococcus aureus, Pseudomonas aeruginosa, Serratia marcescens, Candida albicans, and acanthamoeba keratitis.

In another aspect, the invention provides a lens care solution which has been described above.

In a further aspect, the invention provides a method for cleaning and/or disinfecting a contact lens. The method comprises the steps of: bringing one or more contact lenses into contact with a lens care solution contained in a lens case, wherein either or both of the lens care solution and the lens case comprises a singlet oxygen, wherein if the lens case comprise the singlet oxygen-generating agent, the singlet oxygen-generating agent is covalently attached onto the solution-contacting surface of the lens case; irradiating the singlet oxygen-generating agent for a period of time sufficient to produce a sufficient amount of singlet oxygen in the lens care solution to disinfect the contact lens.

The above described various embodiments of lens care solutions, light irradiation sources, and lens cases can be used in this aspect of the invention.

The solutions and methods of the present invention may be used in conjunction with enzymes to remove debris or deposit material from the contact lens as the solutions of the present invention have no negative effect on the proteolytic activity of enzymes, such as UNIZYME®. After such contacting step, the contact lens optionally may be manually rubbed with saline, or even rinsed without rubbing, to remove further deposit material from the lens. The cleaning method can also include rinsing the lens substantially free of the liquid aqueous medium prior to returning the lens to a wearer's eye.

Although various embodiments of the invention have been described using specific terms, devices, and methods, such description is for illustrative purposes only. The words used are words of description rather than of limitation. It is to be understood that changes and variations may be made by those skilled in the art without departing from the spirit or scope of the present invention, which is set forth in the following claims. In addition, it should be understood that aspects of the various embodiments may be interchanged either in whole or in part. Furthermore, titles, headings, or the like are provided to enhance the reader's comprehension of this document, and should not be read as limiting the scope of the present invention. Accordingly, the spirit and scope of the appended claims should not be limited to the description of the preferred versions contained therein.

### Example 1

P. aeruginosa (9027) is obtained from ATCC and reconstituted in nutrient broth in accordance with ATCC recommendations prior to being frozen in a 10% glycerol solution. P. aeruginosa is grown in nutrient broth for about 18 hours to bring the bacteria into the logarithmic growth phase prior to being spun down via centrifugation and the pellet resuspended in sterile phosphate buffered saline (PBS). The bacterial suspension is then diluted to obtain an OD of 0.9 at 540 nm. This stock bacterial solution is subsequently diluted 1 in 500 to obtain a test solution containing about 10⁵-10⁶ cfu/ml.

Five photosensitizers are used: tetra(N-methyl-4-pyridyl)porphine tetratosylate (TMPyP), Methylene Blue (MB), Toluidine Blue O (TBO) and Rose Bengal (RB) are received from Aldrich (Poole, Dorset, England) and meso-tetra(4-sulfonatophenyl)porphine dihydrochloride (TSPP) is received from Frontier Scientific (Logan, Utah, U.S.A.). A solution of one of the five photosensitizers is made at double the concentration to be tested.

A testing solution (lens care solution) is prepared by adding 5 mL of a photosensitizer solution to 5 mL of the bacterial solution.

Two control solutions are prepared by adding 5 mL of sterile PBS to 5 mL of the bacterial solution. One of the two control solutions is kept in the dark (i.e., Control Dark) and the other one is exposed to the same light conditions as the photosensitizer test solutions (i.e., Control Light).

All solutions (testing and control) are then incubated for about 5 minutes at 37° C. in the dark in an orbital incubator prior to exposure to the relevant light conditions. The light source used provides broad spectrum visible light and the temperature is controlled using a fan. Viable bacteria are then monitored every 15 minutes for the first hour of the experiment and at hourly intervals thereafter to a maximum time of 4 hours.

After incubation overnight, the colony forming units at the dilution where they can best be counted are quantified and number of colony forming units per mL calculated.

The results with testing solutions including TMPyP as sensitizer against about 10⁵ cfu/ml P. aeruginosa are shown in Table 1.

TABLE 1

| | Control | | [TMPyP] (μg/mL) | | |
|---|---|---|---|---|---|
| | Dark | Light | 50 | 250 | 500 |
| Time of Light Exposure (minutes) | 240 | 240 | 45 | 45 | 30 |
| Rate of kill | 0 | 0 | 5-6 | 5-6 | 5-6 |
| (Log reduction in bacterial viability) | | | | | |

A testing solution with 5 μg/mL of TMPyP is also tested against about 10⁵ cfu/ml P. aeruginosa using an alternative light source emitting red light at a wavelength of 630 nm (closer to the λₘₐₓ of absorption of the Q-band of TMPyP) to determine whether irradiating the photosensitizer at such wavelength can maximize singlet oxygen production while limiting the power output of the light source necessary to cause bacterial cell death as well as the concentration of the photosensitizer required. A 2 Log reduction in bacterial viability is observed after one hour of illumination with such a red light.

Where a testing solution with about 1 mg/mLTSPP (close to the TSPP's solubility limit) as sensitizer is tested against about 10⁵ cfu/ml P. aeruginosa, no significant biocidal activity can be observed. TSPP appears to be ineffective against P. aeruginosa.

Where a testing solution with 250 μg/mL TBO as sensitizer is tested against about 10⁵ cfu/ml P. aeruginosa, no reduction in bacterial viability is observed. The results with testing solutions with TBO as sensitizer at two different concentrations against about 10⁵ cfu/ml P. aeruginosa are shown in Table 2.

TABLE 2

| | Control | | [TBO] (μg/mL) | |
|---|---|---|---|---|
| | Dark | Light | 375 | 500 |
| Time of Light Exposure (minutes) | 240 | 240 | 30 | 15 |
| Rate of kill | 0 | 0 | 5-6 | 5-6 |
| (Log reduction in bacterial viability) | | | | |

Where a testing solution with 250 μg/mL MB as sensitizer is tested against about 10⁵ cfu/ml P. aeruginosa, a 1 Log reduction in bacterial viability is observed after four hours of illumination. When the MB concentration in testing solutions is increased to 750 μg/mL and 1 mg/mL respectively, no reduction in viable bacterial is observed after fours hours of light exposure. When the sensitizer RB concentration in a testing solution is about 250 μg/mL, a 5-6 Log reduction in bacterial viability against about 10⁵ cfu/ml P. aeruginosa is observed after 30 minutes of light exposure.

### Example 2

The stock bacterial solution (P. aeruginosa) is prepared according to the procedure described in Example 1 and subsequently diluted 1 in 500 to obtain a test solution containing about 10⁵-10⁶ cfu/ml. The sensitizer solutions (TMPyP, MB and TBO) are prepared according to the procedure described in Example 1. Testing solutions and control solutions are prepared according to the procedures described in Example 1. All solutions are then incubated for about 5 minutes at 37° C. in the dark in an orbital incubator prior to exposure to the relevant light conditions.

Two light sources are used to assess antibacterial activity of the range of photosensitizers. One is a broad spectrum light source which emits light across visible spectrum. This light source produces heat, hence a fan is used to control the temperature of the solutions whilst the rate of kill experiments are ongoing. The other light source used is a red LED, which emits light of a single wavelength at 630 nm. This light source does not emit any heat, hence the temperature does not have to be controlled during the experiments.

Both light sources have comparable power; the broad spectrum light source has a power output of 26.7 mW/cm2. The red LED light source emits with a power of 27 mW/cm2. In both instances, the power is measured using an ILT-1400 A Radiometer/Photometer.

Viable bacteria are monitored every 15 minutes for the first hour of the experiment and at hourly intervals thereafter to a maximum time of 4 hours. After incubation overnight, the colony forming units at the dilution where they can best be counted are quantified and number of colony forming units per mL calculated.

Where testing solutions with TMPyP (5 μg/mL) as sensitizer and the red LED are tested against about 10⁵ cfu/ml P. aeruginosa, a 2 Log reduction in bacterial viability is observed after one hour of light exposure. When the light source is the broad spectrum light source, a 2 Log reduction in bacterial viability against about 10⁵ cfu/ml P. aeruginosa is observed after one hour of light exposure, but a 5-6 Log reduction in bacterial viability is observed after two hours of light exposure. When the TMPyP concentration in a testing solution is increased to 10 μg/mL, a 5-6 Log reduction in bacterial viability against about 10⁵ cfu/ml P. aeruginosa is observed after about 45 minutes of light exposure (either to the red LED or the broad spectrum light source).

Where testing solutions with MB (50 μg/mL) as sensitizer and the broad spectrum light source are tested against about 10⁵ cfu/ml P. aeruginosa, a 3 Log reduction in bacterial viability is observed after one hour of light exposure, and 5-6 Log reduction in bacterial viability is observed after two hours of light exposure.

Where testing solutions with MB (10 μg/mL) as sensitizer and the broad spectrum light source are tested against about 10⁵ cfu/ml P. aeruginosa, a 5-6 Log reduction in bacterial viability is observed after about 45 minutes of light exposure.

Where testing solutions with MB (either at 1 μg/mL or 5 μg/mL) as sensitizer and the red LED light source are against about 10⁵ cfu/ml P. aeruginosa, no reduction in bacterial viability is observed after one hour of light exposure.

Where testing solutions with MB (10 μg/mL) as sensitizer and the red LED light source are tested against about 10⁵ cfu/ml P. aeruginosa, a 3 Log reduction in bacterial viability is observed after about 45 minutes of light exposure, and a 5-6 Log reduction in bacterial viability is observed after about 60 minutes of light exposure.

Where testing solutions with TBO (50 μg/mL) as sensitizer and the broad spectrum light source are tested against about 10⁵ cfu/ml P. aeruginosa, a 3 Log reduction in bacterial viability is observed after being subjected to between 30 to 45 minutes of light exposure.

Where testing solutions with TBO (10 μg/mL) as sensitizer and the broad spectrum light source are tested against about 10⁵ cfu/ml P. aeruginosa, a 5-6 Log reduction in bacterial viability is observed after about 45 minutes of light exposure.

Where testing solutions with TBO (5 μg/mL) as sensitizer and the red LED light source are tested against about 10⁵ cfu/ml P. aeruginosa, a 5-6 Log reduction in bacterial viability against about 10⁵ cfu/ml P. aeruginosa is observed after one hour of light exposure.

Where testing solutions with TBO (5 μg/mL) as sensitizer and the broad spectrum light source are tested against about 10⁵ cfu/ml P. aeruginosa, a 5-6 Log reduction in bacterial viability is observed after about 30 minutes of light exposure.

### Example 3

This example illustrates the determination of uptake of photosensitizers by contact lenses. All photosensitizers are tested at a concentration of 10 μg/mL. For each photosensitizer, five lenses are tested. Experiments are carried out by submerging the lenses in a solution of the photosensitizer and leaving them there overnight. Any excess liquid on the lens surface is removed using medical tissue and any visible color change of the lens is noted. The lenses are also assessed using UV spectrometry in order to quantify any uptake, using a blank lens to provide the baseline for the spectra. The results are shown in Table 3.

TABLE 3

| | AIR OPTIX ® | | ACUVUE ® | |
|---|---|---|---|---|
| | Visual | UV/Vis (μg) | Visual | UV/Vis (μg) |
| TMPyP | invisible | 0 | Highly visible | 5.1 |
| | | | (yellow lens) | |
| MB | invisible | 0.4 | Highly visible | 4.65 |
| | | | (blue lens) | |
| TBO | Slightly | 2.84 | Highly visible | 8.45 |
| | visible | | (blue lens) | |

### Example 4

This example illustrates studies of effects of combination of photosensitizers with hydrogen peroxide (e.g., SoftWear Salne) upon the bacterial viability against S. marcescens.

All materials used and the method used to determine the rate of bacterial kill are described in Example 1 as was the method. In all instances the bacterial challenge is at least 5×10⁵ cfu/mL. SoftWear Saline (providing 0.003% H₂O₂), photosensitizer solutions are made up at double the concentration to be tested using the SoftWear Saline as the diluent. 2.5 mLs of this solution is then mixed with 2.5 mLs of bacteria to form the test solutions.

Where testing solutions with TMPyP (10 μg/mL) as sensitizer and with 0.003% H₂O₂ as microbiocide and a red LED (630 nm) are tested against about 10⁵ cfu/ml S. marcescens, about 3 Log reduction in bacterial viability is observed after one hour of light exposure.

Where testing solutions with MB (10 μg/mL) as sensitizer and with 0.003% H₂O₂ as microbiocide and a red LED (630 nm) are tested against about 10⁵ cfu/ml S. marcescens, a 5-6 Log reduction in bacterial viability is observed after one hour of light exposure. However, when a testing solution contains only MB (20 μg/mL) but is free of H₂O₂, no reduction in bacterial viability against about 10⁵ cfu/ml S. marcescens is observed after one hour of light exposure.

Where testing solutions with TBO (2.5 μg/mL) as sensitizer and with 0.003% H₂O₂ as microbiocide and a red LED (630 nm) are tested against about 10⁵ cfu/ml S. marcescens, a 5-6 Log reduction in bacterial viability is observed after 30 minutes of light exposure. However, when a testing solution contains TBO (5 μg/mL) only but is free of H₂O₂, a 5-6 Log reduction in bacterial viability is observed after 45 minutes of light exposure.

### Example 5

This example illustrates studies of effects of combination of photosensitizers with microbiocide, polyhexamethylene biguanide, PHMB (e.g., Cosmocil CQ solution) upon the bacterial viability against S. marcescens.

All materials used and the method used to determine the rate of bacterial kill are described in Example 1 as was the method. In all instances the bacterial challenge is at least 5×10⁵ cfu/mL. Solutions containing the photosensitizers and PHMB are made up separately at quadruple the concentration to be tested. 1.25 mLs of each solution is then added to 2.5 mLs of bacteria suspension to render the correct concentration to be tested.

Where testing solutions with TMPyP (10 μg/mL) as sensitizer and with 0.0005% PHMB as microbiocide and a red LED (630 nm) are tested against about 10⁵ cfu/ml S. marcescens, a 5-6 Log reduction in bacterial viability is observed after about 15 minutes of light exposure. When the PHMB concentration in a testing solution is decreased to 0.00005% while maintaining TMPyP concentration at 10 μg/mL, a 3 Log reduction in bacterial viability against about 10⁵ cfu/ml S. marcescens is observed after about 45 minutes of light exposure.

Where testing solutions with MB (10 μg/mL) as sensitizer and with 0.0005% PHMB as microbiocide and a red LED (630 nm) are tested against about 10⁵ cfu/ml S. marcescens, a 5-6 Log reduction in bacterial viability is observed after about 15 minutes of light exposure. When the PHMB concentration in a testing solution is decreased to 0.00005% while maintaining MB concentration at 10 μg/mL, more than 3 Log reduction in bacterial viability against about 10⁵ cfu/ml S. marcescens is observed after one hour of light exposure.

Where testing solutions with TBO (2.5 μg/mL) as sensitizer and with 0.0005% PHMB as microbiocide and a red LED (630 nm) are tested against about 10⁵ cfu/ml S. marcescens, a 5-6 Log reduction in bacterial viability is observed after about 15 minutes of light exposure. When the PHMB concentration in a testing solution is decreased to 0.00005% while maintaining TBO concentration at 2.5 μg/mL, a 5-6 Log reduction in bacterial viability against about 10⁵ cfu/ml S. marcescens is observed after about 30 minutes of light exposure. Where the PHMB concentration and TBO concentration in a testing solution are 0.00005% and 1.25 μg/mL respectively, a 3 Log reduction in bacterial viability against about 10⁵ cfu/ml S. marcescens is observed after about 15 minutes of light exposure, and a 5-6 Log reduction in bacterial viability against about 10⁵ cfu/ml S. marcescens is observed after about 45 minutes of light exposure. Where the PHMB concentration and TBO concentration in a testing solution are 0.00005% and 0.625 μg/mL respectively, a 3 Log reduction in bacterial viability against about 10⁵ cfu/ml S. marcescens is observed after about 15 minutes of light exposure, and a 5-6 Log reduction in bacterial viability against about 10⁵ cfu/ml S. marcescens is observed after about 30 minutes of light exposure to a LED (630 nm).

As a control, experiments with a testing solution containing 0.00005% PHMB and without any sensitizer, only a 1 Log reduction in bacterial viability against about 10⁵ cfu/ml S. marcescens is observed after one hour.

Where a testing solution contains 10 μg/mL RB and is free of H₂O₂ or PHMB, no reduction in bacterial viability against about 10⁵ cfu/ml S. marcescens is observed after light exposure to a LED (630 nm). Where a testing solution contains 10 μg/mL RB and 0.003% H₂O₂, no reduction in bacterial viability against about 10⁵ cfu/ml S. marcescens is observed after light exposure to a LED (630 nm). Where a testing solution contains 10 μg/mL RB and 0.00005% PHMB, only a 2 Log reduction in bacterial viability against about 10⁵ cfu/ml S. marcescens can be observed after 15 minutes of light exposure to a LED (630 nm) and additional light exposure up to one hour does not reduce bacterial viability against about 10⁵ cfu/ml S. marcescens.

Where a testing solution contains 10 μg/mL TMPyP and 0.00005% PHMB, a 5 Log reduction in bacterial viability against about 10⁷ cfu/ml C. albicans is observed after one hour of light exposure to a LED (630 nm).

### Example 6

This example illustrates the determination of uptake of photosensitizers in a lens care solution by various commercial contact lenses. Lens care solutions are prepared according to the procedure described in Examples 4 and 5 to having the composition shown in Table 4.

TABLE 4

| | [Photosensitizer] (μg/mL) | | | [Microbicide] (by weight) | |
|---|---|---|---|---|---|
| | TMPyP | MB | TBO | H2O2 | PHMB |
| LCP-1 | 10 | | | 0.03% | |
| LCP-2 | | 10 | | 0.03% | |
| LCP-3 | | | 2.5 | 0.03% | |
| LCP-4 | 10 | | | | 0.00005% |
| LCP-5 | | 10 | | | 0.00005% |
| LCP-6 | | | 0.3125 | | 0.00005% |

Experiments are carried out by submerging lenses in a lens care solution and leaving them there overnight. Any excess liquid on the lens surface is removed using medical tissue and any visible color change of the lens is noted. The lenses are also assessed using UV spectrometry in order to quantify any uptake, using a blank lens to provide the baseline for the spectra. The results are shown in Table 5.

TABLE 5

| | LCP-1 | LCP-2 | LCP-3 | LCP-4 | LCP-5 | LCP-6 |
|---|---|---|---|---|---|---|
| Acuvue 2 | Strongly | Strongly | Strongly | Strongly | Strongly | No tint |
| | yellow tinted | blue tinted | blue tinted | yellow tinted | blue tinted | |
| | 3.15 μg/mL | 1.89 μg/mL | 1.74 μg/mL | 3.24 μg/mL | 1.83 μg/mL | 0 |
| Acuvue | Weakly | No tint | Weakly blue | Weakly | No tint | No tint |
| Oasys | yellow tinted | | tinted | yellow tinted | | |
| | 1.14 μg/mL | 0 | 0.33 μg/mL | 0.98 μg/mL | 0 | 0 |
| Air Optix | No tint | No tint | Weakly blue | No tint | No tint | No tint |
| | | | tinted | | | |
| | 0.72 μg/mL | 0 | 0.31 μg/mL | 0.54 μg/mL | 0 | 0 |

What is claimed is:

1. A lens care solution for disinfecting and/or cleaning contact lenses, comprising:
(1) a singlet oxygen-generating agent which is dissolved or dispersed in a lens care solution; and
(2) one or more components selected from the group consisting of buffering agent(s), lubricant(s), conditioning/wetting agent(s), viscosity-enhancing agent(s), tonicity agent(s), surfactant(s), chelating agent(s), microbicide(s), preservative(s), and combinations thereof.

2. The lens care solution of claim 1, wherein the singlet oxygen-generating agent is Rose Bengal, methylene blue, Azure A, a porphyrin, a metalloporphyrin, a phthalocyanin, a metallophthalocyanin, or combinations thereof.

3. The lens care solution of claim 1, wherein the lens care solution has at least one property selected from the group consisting of a pH of from about 6.0 to about 8.0, a tonicity of from about 200 to about 450 milliosmol (mOsm), a viscosity of from about 1.0 centipoise to about 20 centipoise at 25° C., and combinations thereof.

4. The lens care solution of claim 1, wherein the singlet oxygen-generating agent is dissolved or dispersed in the lens care solution.

5. The lens care solution of claim 4, wherein the singlet oxygen-generating agent is covalently attached to the surfaces of particles which are dispersed in the lens care solution.

6. The lens care solution of claim 4, wherein the singlet oxygen-generating agent is covalently attached to a hydrophilic polymer having a molecular weight sufficient high so as to prevent the singlet oxygen-generating agent from being absorbed by the contact lens under disinfection based on size alone, wherein the hydrophilic polymer with the singlet oxygen-generating agent attached thereon is dissolved or dispersed in the lens care solution.

7. The lens care solution of claim 4, wherein the lens care solution comprises at least one buffering agent selected from the group consisting of boric acid, sodium borate, potassium borate, citric acid, sodium citrate, potassium citrate, potassium bicarbonate, sodium bicarbonate, TRIS (trometamol, 2-amino-2-hydroxymethyl-1,3-propanediol), bis-aminopolyols, Na₂HPO₄, NaH₂PO₄, K₂HPO₄, and KH₂PO₄ or mixtures thereof, wherein the amount of the buffer agent is necessary to be effective in achieving a pH of from about 6.5 to about 7.5.

8. The lens care solution of claim 4, wherein the lens care solution comprises one or more components selected from the group consisting of polyvinylalcohol, polyvinylpyrrolidone, a vinylpyrrolidone/vinylacetate copolymer, a vinylpyrrolidone/dimethylaminoethylmethacrylate copolymer, a vinylpyrrolidone/acrylic acid copolymer, a vinylpyrrolidone/methacrylic acid copolymer, a vinylpyrrolidone/vinylcaprolactam/dimethyl-aminoethylmethacrylate copolymer, a alkylated polyvinyl pyrrolidone copolymer, a vinylpyrrolidone/vinyl acetate copolymer with a given degree of hydrolysis (e.g., at least a degree of hydrolysis of at least about 70%, preferably at least about 80%, even more preferably at least about 90%), methyl cellulose (MC), ethyl cellulose, hydroxymethylcellulose, hydroxyethyl cellulose (HEC), hydroxypropylcellulose, hydroxypropylmethyl cellulose (HPMC), hyaluronic acid or salts thereof, carboxymethylcellulose, polyglycolic acid, polylactides, collagen, gelatin, xanthan gum, gum Arabic, starch, polyacrylic acid, polymethacrylic acid, copolymer of acrylamide and acrylic acid, and combinations thereof.

9. The lens care solution of claim 4, wherein the lens care solution comprises one or more tonicity agents selected from the group consisting of sodium chloride, potassium chloride, glycerol, propylene glycol, polyols, dexpanthenol, mannitols, xylitol, sorbitol, and mixtures thereof.

10. The lens care solution of claim 4, wherein the lens care solution comprises a surfactant selected from the group consisting of homopolymers of polyethylene glycol or polyethyleneoxide, poloxamers which are nonionic surfactants consisting of block copolymers of propylene oxide and ethylene oxide; poloxamine which is a block copolymer derivative of ethylene oxide and propylene oxide combined with ethylene diamine; tyloxapol which is 4-(1,1,3,3-tetramethylbutyl)phenol polymer with formaldehyde and oxirane; ethoxylated alkyl phenols; polysorbates; alkyl glucosides and polyglucosides; polyethoxylated castor oils; and combinations thereof.

11. The lens care solution of claim 4, wherein the lens care solution comprises at least one microbicide.

12. The lens care solution of claim 4, wherein the lens care solution comprises at least one microbicide selected from the group consisting of biguanides and salts thereof, biguanide polymers and salts thereof, a polyquaternium, bronopol, benzalkonium chloride, hydrogen peroxide, and combinations thereof.

13. The lens care solution of claim 4, wherein the lens care solution comprises a hexamethylene biguanide polymer in an amount of from about 0.05 to about 5 ppm.

14. The lens care solution of claim 4, wherein the lens care solution comprises a hexamethylene biguanide polymer in an amount of from about 0.2 to about 1.0 ppm.

15. A lens care kit comprising a lens care solution of claim 1, wherein the lens care kit further comprises: a lens case for holding the lens care solution and a contact lens immersed in the lens care solution; and a light irradiation source for irradiating the singlet oxygen-generating agent for a period of time sufficient to produce a sufficient amount of singlet oxygen in the lens care solution to disinfect the contact lens.

16. The lens care kit of claim 12, wherein the lens case comprises the light irradiation source which is a light emitting device (LED).

17. The lens care kit of claim 13, wherein the LED would turn on inside the lens case after the lens case cap(s) for the lens case is (are) placed into place in a sealed state.

18. A method for cleaning and/or disinfecting a contact lens, comprising the steps of:
(1) bringing one or more contact lenses into contact with a lens care solution contained in a lens case, wherein either or both of the lens care solution and the lens case comprises a singlet oxygen-generating agent, wherein if the lens case comprise the singlet oxygen-generating agent, the singlet oxygen-generating agent is covalently attached onto the solution-contacting surface of the lens case; and
(2) irradiating the singlet oxygen-generating agent for a period of time sufficient to produce a sufficient amount of singlet oxygen in the lens care solution to disinfect the one or more contact lenses.
